(19) United States
(12) Patent Application Publication
(10) Pub. No.: US 2018/0078413 A1
(43) Pub. Date: Mar. 22, 2018

(54) PHACOEMULSIFICATION DEVICE WITH PRESSURE FEEDBACK

(71) Applicant: Richard S. KOPLIN, New York, NY (US)

(72) Inventor: Richard S. KOPLIN, New York, NY (US)

(21) Appl. No.: 15/823,178

(22) Filed: Nov. 27, 2017

Related U.S. Application Data
(63) Continuation of application No. PCT/US16/34607, filed on May 27, 2016.
Application No. PCT/US16/34607 is a continuation of application No. 15/165,736, filed on May 26, 2016, now abandoned.
(60) Provisional application No. 62/166,821, filed on May 27, 2015.
(60) Provisional application No. 62/166,821, filed on May 27, 2015.

(51) Int. Cl. A61F 9/007; A61M 1/00; A61M 5/158; A61M 3/02

(52) U.S. Cl. CPC A61F 9/00745; A61M 1/0064; A61M 5/158; A61M 3/0283; A61M 2205/3592; A61M 2205/3576; A61M 2205/3344; A61M 2205/0244; A61M 3/0237; A61M 2205/8206; A61M 2205/8237; A61M 2210/0612; A61F 9/00781

(57) ABSTRACT

A phacoemulsification system includes a platform controlling the phacoemulsification process and a handpiece attached to the platform and used by a surgeon to perform an operation. The handpiece includes a hollow phaco needle with a flexible sleeve around the needle and provides an infusion fluid sourced at the platform into the eye. The sleeve, which may be disposable, is provided with a sensor device configured to sense the instantaneous fluid pressure in the eye during the operation and send this information to the platform. This information is used by the process to control the pressure of the infusion fluid to insure that the eye is not injured and to otherwise protect the eye during the operation.

### CROSS-REFERENCE TO RELATED APPLICATION

This application is a continuation of PCT/US16/34607, filed May 27, 2016, which claims priority to U.S. Provisional Application Ser. No. 62/166,821 filed May 27, 2015, the disclosure of each of which is incorporated herein by reference in its entirety.

### BACKGROUND

Modern cataract surgery typically utilizes an integrated mechanical and fluidics system supporting a surgeon hand-piece whose front-end houses a hollow needle surrounded by a soft sleeve typically of a silicone material. The hollow needle provides (usually) ultrasonic vibrations used to emulsify the cataractous lens. Infusion fluid (physiologic saline) enters the eye and is used as a medium for transport of the fragmented lens material to the tip of the hollow ultrasound needle. This infusion fluid runs down the surgeon's hand-piece between the ultrasonic needle (measuring about 0.9 mm in diameter) and the surrounding sleeve. This infusion fluid is delivered through distal ports (typically two, positioned 180 degrees from one another) on the sleeve into the anterior chamber of the eye filling the space, maintaining the vault and thus preventing collapse of the cornea or iris against the phaco needle/sleeve complex during the emulsification process. The infusion fluid and the lens material are aspirated from the eye through the hollow needle and into a waste receptacle on the system platform.

Balancing fluid infusion into the eye against its exit from the eye via aspiration through the hollow needle and passive leakage around the surgical entry wound has long been a concern of the industry. As a general term the performance of fluid infusion into the eye is referred to as fluidics.

Recently the industry—both surgeons as end users and manufacturers—have come to realize that both the safety and efficacy of phacoemulsification surgery is associated with previously unknown or under-appreciated risks, as demonstrated by a number of peer reviewed publications. These findings include cataractous lens material accumulating in the posterior portion of the eye as well as trauma to the iris. Both of these phenomena may cause inflammation in the post-operative period and, secondarily, diminish visual potential.
1. Novak, J, Retrolenticular Lens Fragments: a problem of phacoemulsification; J. Ev. Purkyne Praha: 1999 September; 55(5): 268-273
2. Ang A, Menezo i Rallo V, Shepstone L, Burton R L. Retrocapsular lens fragments after uneventful phacoemulsification cataract surgery, J Cataract Refract Surg 2004; 30:849-853
3. Koplin R S, Ritterband D C, Dodick J M, Donnenfeld E D, Schafer, M: (in press) Untoward events associated with aberrant fluid infusion during cataract surgery: a laboratory study with corroborative clinical observations.
4. Chang D F, Campbell J R. Intraoperative floppy iris syndrome associated with tamsulosin. J Cataract Refract Surg. 2005; 31:664-73.
5. Oh J H, Chuck R S, Do J R, Park C Y. Vitreous Hyper-Reflective Dots in Optical Coherence Tomography and Cystoid Macular Edema after Uneventful Phacoemulsification Surgery Published: PLOS ONE 2014; 9:1-9

These risks are directly related to inconsistent regulation of infusion fluid entering the eye, admixing with lens material, and adversely impacting intraocular anatomy. Importantly, the volume of fluid delivered into the eye at any given time also affects the control of the procedure. The result of unregulated fluid infusion is variable and unpredictable intraocular pressure that relates to constant fluctuations in anterior chamber depth and increased turbulence and inertial forces—facilitating a higher Reynolds Number. This process compromises the ability to maintain lens material at the phaco tip for efficient emulsification and promoting untoward lens transport to the back of the eye and aggravation the iris causing disquieting and damaging flapping movements of the iris. (Often called Floppy Iris Syndrome.) These phenomena affect both the efficiency and safety of the operation.

Fluid infusion into the eye during phacoemulsification surgery is historically provided by gravity feed. Typically, a bottle of a physiologic saline solution is positioned at approximately 60-120 cm above the eye and tubing from the bottle is connected through a surgical hand-piece to the needle/sleeve complex where fluid leaving the eye—through aspiration defined by a linear process (surgeon presses the foot-pedal to increase aspiration flow rate of fluid) and leakage from various wounds in the eye will commensurately dictate the rate of fluid infusion. The average the rate of infusion into an eye at surgery is about 30 cc/minute, but can vary widely depending on the aspiration rate, leakage from surgical wounds, and the size of the eye as well as certain preset platform functions, including bottle height.

The negative characteristics of a gravity feed fluid infusion system is the sudden impact on the anatomy as unrestrained fluid enters the eye as the surgeon engages a position on the system foot pedal to initiate infusion. Also to contend with are fluid surges when vacuum is relieved at the needle tip once a piece of cataractous lens (at first blocking aspiration into the hollow needle) is suddenly aspirated into the hollow needle causing vacuum to fall abruptly. Depending on the size of the eye and the stability of the lens to be emulsified infusion fluid entering the eye in unregulated fashion may cause an alarming deepening of the anterior chamber, forcing the lens posteriorly, causing the patient pain, and making surgery more risky. In other cases, where the eye is small, fluid infusion may be forced into constrained anatomical compartments where this crowding may cause disconcerting movements of the iris out of the surgical wounds. Secondary to this blockage the anterior chamber may become abruptly shallower in the presence of an unrestricted inflow of the fluid, requiring the surgeon to remove the instruments perform emergency measures to relieve the high pressure disrupting the procedure.

In order to mitigate the damaging effects of unregulated fluid infusion into the eye—and attempting to balance this with aspiration and leakage—the surgeon is required to empirically decrease or increase preset platform values for aspiration and/or place the infusion bottle at varying heights (lower the bottle to attempt to decrease the volume of infusion entering the eye or raise the bottle in order to increase infusion). Because of these concerns there is a growing acknowledgement that a form of active controls is required maintain control of fluid infusion at phaco surgery.

Active fluidics, that is the process of controlling fluid infusion utilizing control mechanisms responsive to changes in intraocular pressure, is essentially a sensor regulated process that is designed to respond to monitored changes in eye pressure. However, a major failing of existing active fluidic designs is that there is a lag time between a call for a response to changes in fluid pressure within the eye because the sensor is positioned down the line (responding via fluid pressure within a flexible tubing transporting fluid into the eye and installed within the tubing or at its terminus in the platform several feet away). The delay significantly impacts the system's ability to engage the mechanism (peristaltic pump, gas, Venturi) facilitating the response to adequately provide and accurate amount of fluid flow into the eye. Because of this lag-time the benefits to patients and surgeons provided by the present state of active fluidics is significantly degraded. So, in spite of the active fluidics presently installed in certain platforms, the surgeon still experiences extreme surges of infusion impacting the contents of the eye, resulting in turbulence and visibly driving cataractous lens particles about the eye—in both anterior and posterior segments.

Intraocular pressure measurements (fluid eye pressure measurements) have been a staple for understanding the health of the eye for many years. Elevated intraocular fluid pressure (usually measured in mm Hg) was not routinely assessed until the latter part of the 19th century when von Graefe developed the first instrument for measuring intraocular pressure in (1865).

Intraocular pressure is typically measured to aid in the monitoring and treatment of a disorder called glaucoma, where an untoward elevation in the eye pressure may result in damage to the optic nerve and impact vision. Investigators have long imagined implanting permanent sensors in eye tissues to provide real-time monitoring of eye pressure and allowing more timely treatment of a patient's glaucoma.

### SUMMARY

It is clear that in order to provide a real time monitor for an active fluidics system a sensor must be within the anterior chamber and wirelessly disposed where it can provide real time information to a (trans)receiver on the surgical platform a few feet away. Only in this way can the monitor maintain a timely response for adequate control of fluid infusion into the eye.

The present invention is not fixed within the eye (not implanted), is not attached or adherent to any eye tissues; and is not directed to measure eye pressure for disease prevention or treatment. Infusion fluid (typically a physiologic saline solution) is provided directly from a source in the surgical platform (via a pump: peristaltic, or Venturi, or gas) through a flexible tubing into the phaco needle/sleeve complex incorporated within a hand-piece within the eye during phaco surgery. There is a measurable fluid pressure within the eye (specifically for our purposes a measure within the anterior chamber) that dictates the environmental stability of the anterior and posterior chambers. In order to produce a beneficial homeostatic fluid environment during phaco surgery, the intraocular pressure must be regulated thus producing a predictable balance of fluid infusion entering the eye as well as exiting the eye. The basis for this balancing process is the pressure within the eye at any given instant. This pressure must be measured in real-time for the regulation to be effective. In the present invention, this pressure is measured and communicated (preferably wirelessly) to a receiving device within the phaco platform for real-time control of the servo mechanism controlling fluid inflow to the eye.

Thus, the invention is designed to provide a temporary monitoring of eye pressure during the phacoemulsification surgery, with a sensor being placed on or within the disposable silicone sleeve surrounding the phaco needle. The sensor provides a relative or absolute pressure reading of the intraocular pressure during surgery, in the form of a continuous data stream that can be monitored at the terminus of the fluid line from the eye at the system platform. This data can be utilized by the surgeon's to preset controls at the servo pump or gas infusion device within the platform in order to maintain a constant pressure environment within the eye during surgery. This information from the sensor within the eye and on or in the sleeve is preferably transmitted to a relay or repeater near the patient's eye and from there to a transceiver positioned within the surgical platform. The surgical platform houses the mechanism for servicing a call for infusion using the differential between the platform preset target pressure (as determined by the surgeon) and the actual intraocular pressure as determined by the sensor situated in or on the sleeve. The mechanism is meant to call for fluid infusion (or not) when the data stream suggests a there is a differential in the preset and actual intraocular pressure. Thus a control loop is provided to operate as a real-time active fluidics mechanism as if the fluid environment within the eye, the aspiration line, and the infusion line were continuous and in a state of equilibrium.

The sensor, attached to the sleeve can be rendered inoperable once the surgical instruments—along with the sensor—are removed from the eye at the conclusion of the operation.

In one embodiment, the sensor is implemented as an RFID disposed on a chip using, for example, a silicon-on-insulator process. This configuration is advantageous because it provides a practical system architecture with low power and size, characteristics that are advantageous for the present invention.

One embodiment of the present invention includes a micro-, or nano sensor powered sensing and amplification device that is provided as a package for placement outside or within the wall of a silicone sleeve specific to the infusion process for phacoemulsification surgery.

In one embodiment of the present invention, the sensor is disposed on a second instrument that is temporarily inserted into the eye during the phacoemulsification procedure and then is removed, and, optionally disposed of at the conclusion of surgery.

Another embodiment of the present invention provides for the temporary placement of the sensor through the corneal tissue to enter the eye at minimum where only the sensor is exposed to the intraocular environment Another embodiment of the present invention provides for a system wherein the flow of infusion fluid is used as a source of energy inside the eye.

Another embodiment of the present invention includes a power supply configured as a radio frequency receiver and powered externally.

Another embodiment of the present invention provides an antenna configured as a wireless source of data transmission Another embodiment of the present invention provides the envisioned system as having a storage unit as a source of reliable power.

Another embodiment of the present invention provides a capacitor as is utilized as a power storage unit.

Another embodiment of the present invention utilizes a battery configured as the power storage unit.

Another embodiment of the present invention provides for such a system where the sensor package continuously monitors and transmits data from the eye to the external transceiver and then to the platform console for instantaneous response by a servo mechanism to control infusion fluid.

Another embodiment of the present invention provides for such a system where the sensor package continuously monitors and transmits data from the eye to the external transceiver and then to the platform console for real-time response by a servo mechanism to control infusion fluid as well to a responsive transceiver coordinating the aspiration process where a fine balance between the two can further maintain intraocular pressure.

Furthermore, the sensor-sleeve complex is designed for temporary use and once removed from the eye can be disposed of, or in certain cases, re-sterilized for multiple use.

Another embodiment of the present invention the pressure sensor is a micro-electro-mechanical (MEMS) pressure sensor.

Another embodiment of the present invention provides that an RFID chip with the sensor built into the chip and an external source querying the RFID chip also provides the energy for the sensor.

The features and advantages described herein are not all-inclusive and, in particular, many added features and advantage will be apparent to one of ordinary skilled in the art and conversant with the drawings, specifications, and claims. Moreover it should be noted that the language used in the specification has been purposefully selected for readability and instructional purposes, and not to limit the scope of the inventive subject.

### BRIEF DESCRIPTION OF THE DRAWINGS

FIG. 1 shows a prior art phacoemulsification system with gravity fed infusion;

FIG. 2 shows a prior art phacoemulsification system with a liquid infusion pump;

FIG. 3 shows a block diagram a phacoemulsification system a liquid infusion pump and a sensor constructed in accordance with this invention;

FIG. 4 shows an alternate embodiment of the system of FIG. 3 using an air pump for pressurizing the infusion fluid;

FIG. 5 shows a circuit diagram for a sensor device used in the phacoemulsification systems of FIG. 3 or 4;

FIG. 6 shows a circuit diagram of an alternate embodiment for the sensor device.

### DETAILED DESCRIPTION

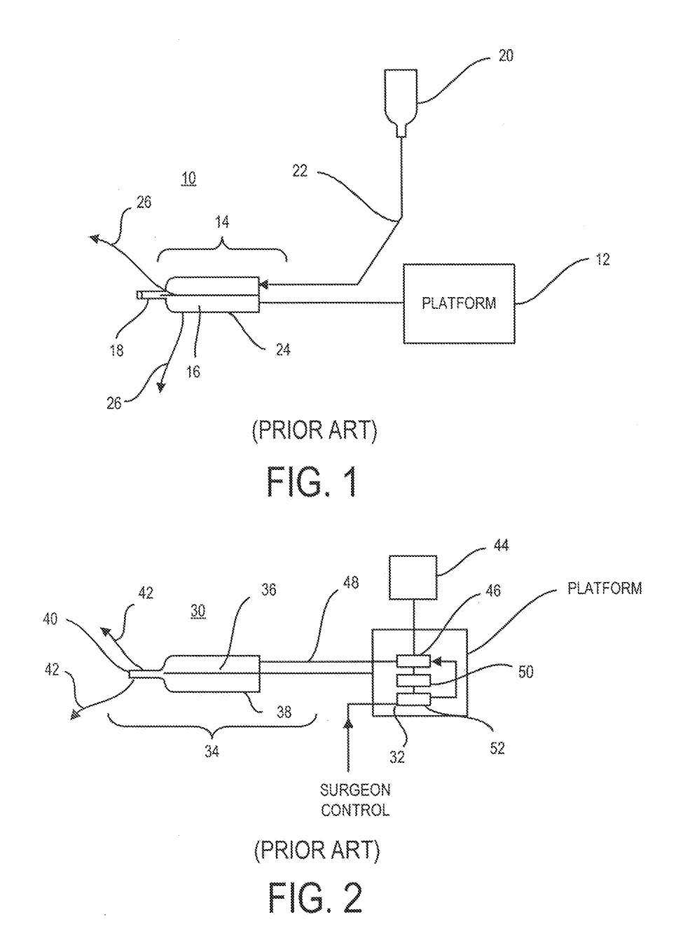

Referring to FIG. 1, a first conventional phacoemulsification system 10 includes a platform 12 is associated with a handpiece 14. The handpiece 14 includes a hollow needle 16 that is connected to the platform 12 and is vibrated at known ultrasonic frequencies so that when its tip 18 is inserted into proximal to a cataractous lens within the eye (not shown), it can emulsify the lens contained therein. A bottle 20 provides through a tube 22 an infusion fluid that flows around the needle 16 through a sleeve 24 and exits, as at 26 adjacent to the tip 18. The pressure of the fluid at the exit points 26 can be controlled in this system 10 only by raising or lowering the bottle 20.

A somewhat more sophisticated system 30 is shown in FIG. 2. System 30 includes a platform 32 that is connected to handpiece 34 also having a hollow needle 36 surrounded by a sleeve 38 and having an emulsification tip 40. Infusion fluid is provided from the platform 32 to the sleeve 38 and this fluid is then ejected near tip 40, as at 42. In this system, the infusion fluid is provided by a bag 44 to a pump 46. The pump 46 then forces the fluid through tube 48 to the sleeve 38. The platform 32 also includes a sensor 50 that senses the fluid pressure through the line (tubing) as it leaves the pump 46. The operation of the pump 46 is controlled by a control servo 52 which receives an input from the surgeon indicative of the desired fluid pressure and another input from the sensor 50. ideally, this feedback-type control scheme should be able to control the pressure of the fluid as it is ejected at 42. However, in practice it has been found that this scheme is less than ideal because there has a substantial and measurable lag between the time that surgeon sets a desired pressure demand as an input for the servo and the time the loop adjusts itself to the desired pressure. As a result, the pressure at the fluid exit points 52 can be either much higher or much lower than desired for a considerable time, leading to complications within the eye.

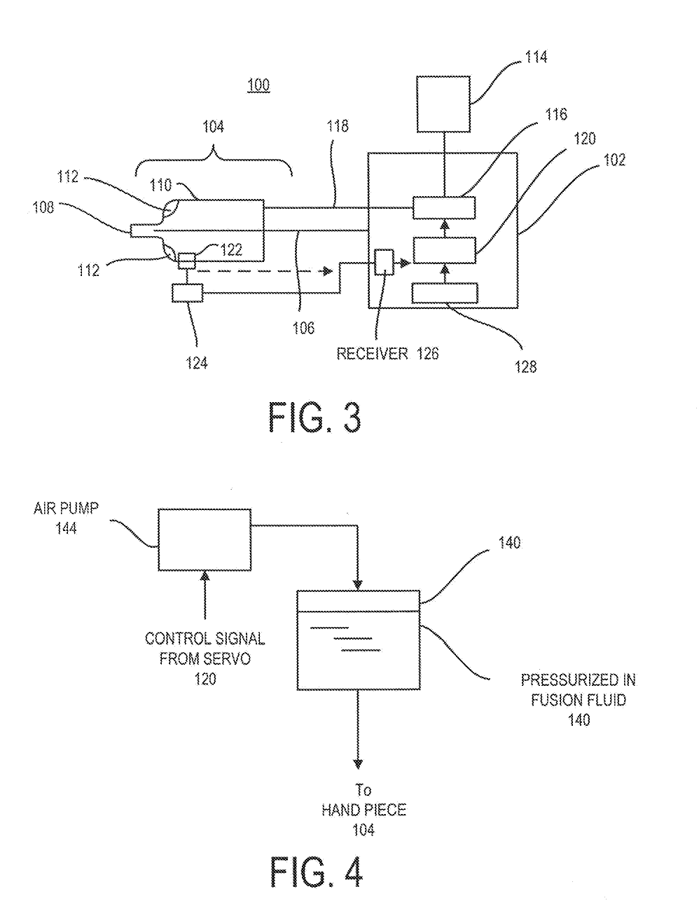

A system 100 constructed in accordance with this invention is shown in FIG. 3. As shown in this Figure, the system 100 includes a platform 102 associated with a handpiece 104. The handpiece includes a needle 106 terminating a tip 108 used for emulsification. A sleeve 110 surrounds the needle 106 and provides fluid near the tip 108 through exit ports 110. The needle 106 is vibrated at ultrasonic frequencies by a conventional ultrasonic generator disposed in the platform 102 (not shown). The sleeve may be disposable.

The infusion fluid originates from a bag 114 and is pushed by a pump 116 through fluid tube 118. The pump 116 is controlled by a servo 120. Importantly, a sensor device 122 is disposed adjacent to one of the exit ports along or in the sleeve 112. The sensor device 122 is arranged to measure the instantaneous fluid pressure at that point. The pressure information from sensor device 122 is transmitted to an information relay (such as an RF transceiver or repeater) 124. The relay 124 then transmits the pressure information to a receiver 126 in the platform 102. The pressure information is then provided to a servo 120. The platform 102 is also provided with a surgeon interface 128 that receives demand information from the surgeon. The interface 128 may include a dial or a digital keypad used by the surgeon to set a certain fluid pressure or request a pressure increase or decrease. The servo 120 then uses the pressure information and a demand signal from the interface 128 to control the operation of the pump 116. Since the pressure information originates directly from the fluid exit port 112, it is much more accurate or current then in the prior art and hence the system 130 operates much faster and more reliably.

In one embodiment of the invention, instead of using relay 124, the pressure information from the sensor device 122 is transmitted to the receiver 126 by a hard wire, an RF transmission, etc.

Sensor device 122 is preferably a miniaturized IC chip that can be mounted at a location preferably near one of the exit ports 112. For example, device 122 can be mounted on the inner or outer wall of sleeve 110. The information relay or repeater 124 is disposed preferably outside the eye but near enough so that it can be within the transmitting range of device 122.

In the embodiment described above, the infusion fluid for the handpiece 104 is pressurized directly and controlled using a pump 116, which may be, for example, a peristaltic pump. In an alternate embodiment, instead of using a direct pressurizing means, an air (or gas) pump may be used, as shown in FIG. 4. In this Figure, a pressurized vessel 140 holds an infusion fluid 142. This fluid 142 is fed to the handpiece 104 as described in conjunction with FIG. 3. The vessel 140 is pressurized by an air pump 144 that is operated by a control signal from the servo 120.

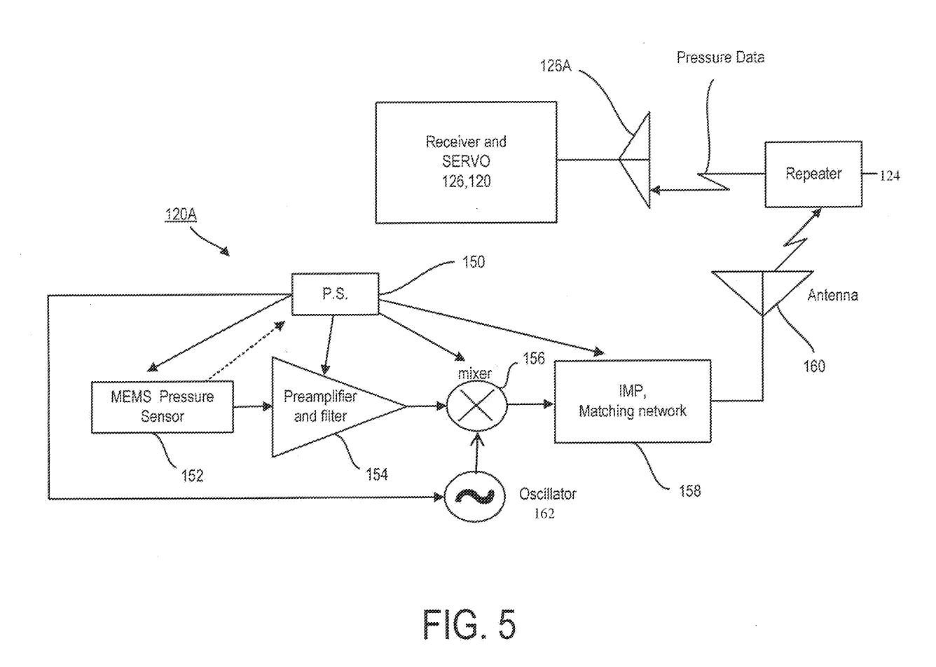

FIG. 5 shows a block diagram of a first embodiment 120A of the sensor device. It includes a power supply 150, a sensor element 152, a preamplifier and filter 154, a mixer 156, an impedance matching network 158 and an antenna 160.

The sensor element 152 is preferably a MEMS-type pressure sensor in communication with the infusion fluid within or exiting from the sleeve 110. The sensor output is conditioned and amplified by preamplifier and filter 154 and fed to a mixer 156. The mixer 156 further receives an RF signal from a local oscillator 162. The resulting RF signal is fed to an impedance matching network 158 and output by antenna 160.

While the output signal from the antenna could be transmitted straight to the platform 102, or it can be transmitted through a repeater 124 as discussed above. The RF output signal is received by an antenna 126A incorporated into platform 102 and then fed to the receiver 126 and servo 120 as discussed above.

The power supply 150 can be either a battery, a supercapacitor or other conventional static power source. Alternatively, power to the sensor device 122 can be provided by an active source. For example, in FIG. 5 such as an inductor 170 is provided in an external excitation member 172. Excitation member 172 is disposed outside the eye during surgery and is provided power from an external power source 174. During surgery, the inductor 170 generates a magnetic field that in turn generates a current through internal inductor 176. The inductor 176 with a capacitor 178 form an active power source 150A for the sensor device shown in FIG. 4.

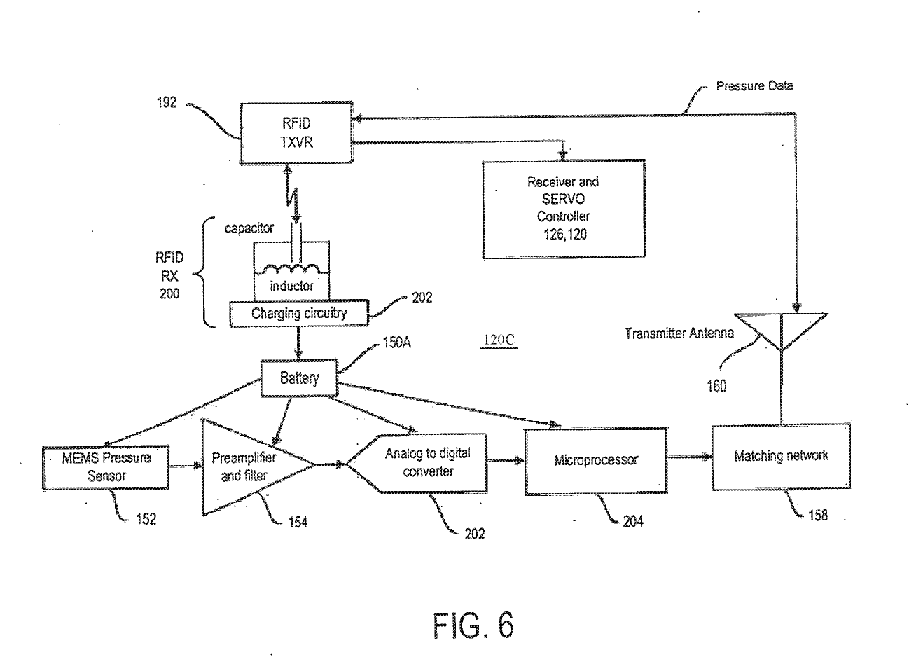

FIG. 6 shows several other alternative embodiments. In this Figure sensor 120C is a digital device as opposed to the analog device shown in FIG. 4. The sensor element 152 generates sensor data that, after processing by the amplifier and filter 154 is provide to an ADC 202. The digital sensor data from the ADC is sent out either directly or fed to a microprocessor 204. The microprocessor then generates corresponding digital output data that is fed to the impedance matching network 158 and then to antenna 160. In one embodiment this output data is sent either directly to the receiver of the platform 102 either directly, or via repeater 124. In this configuration, power for the sensor device 120C is provided by battery 150A.

In another embodiment, RFID technology is used to query and power the sensor device 120C. For this purpose, an external RFID transceiver 192 is provided that is positioned during surgery adjacent to the surgery site. The sensor device 120C includes an RFID receiver and tank circuit 200 feeding a charging circuit 202. When activated, the RFID transceiver sends a query to the RFID receiver 200 in the form of an RF signal. This RF signal is preferably continuous.

The RFID receiver 200 receives the RF signal and uses its energy to power a charging circuit 202. The charging circuit then generates power that is either used to energize the other elements of the device 120C directly, or is used to charge battery 150A. Then, in response to the query, the sensor element detects the respective fluid pressure and generates a corresponding output signal indicative of this instantaneous fluid pressure. In one embodiment, the output signal from the antenna 160 is transmitted to the platform 102 directly or via repeater 124. In another embodiment, the RFID external transceiver 192 also acts as the repeater 124. In this case, the antenna 160 is part of the RFID receiver 200 and the output signal is sensed by the transceiver 192 which then transmits it to the platform 102.

Numerous modifications may be made to this invention without departing from its scope as defined in the appended claims.

What is claimed is:

1. A phacoemulsification apparatus for performing surgery within a capsular chamber of an eye of a patient, comprising:
a handpiece having a hollow needle adapted to emulsify cataractous lenses, a sleeve disposed about the needle and having an input adapted to receive infusion fluid to irrigate the capsular chamber during the surgery;
a platform adapted to provide the infusion fluid and being responsive to a sensor signal;
a sensor device being sized to be disposed within the capsular chamber and to generate the sensor signal, the sensor signal being indicative of a current fluid pressure within the capsular chamber, and the sensor device including an RFID receiver; and
an external RFID transceiver adapted to be disposed adjacent to the sensor device to receive the sensor signal through the RFID receiver, the external RFID transceiver being adapted to transmit the sensor signal to the platform.

2. The apparatus of claim 1, wherein the sensor device includes a MEMS device.

3. The apparatus of claim 1, wherein the sensor device is disposed on the sleeve.

4. The apparatus of claim 1, wherein the sensor device is coupled to an inner surface of the sleeve.

5. The apparatus if claim 1, wherein the sensor device is coupled to an outer surface of the sleeve.

6. The apparatus of claim 1, wherein the platform includes a pump adapted to pump the infusion fluid to the sleeve input.

7. The apparatus of claim 6, wherein the platform is adapted to receive and respond to a demand signal from a surgeon using the apparatus.

8. The apparatus of claim 1, wherein
the infusion fluid is disposed in a pressurized vessel, and
the platform includes an air pump adapted to selectively pressurize the pressurized vessel in response to a control signal and the sensor signal.

9. The apparatus of claim 1, wherein the sensor device includes a power supply adapted to provide power to the sensor device.

10. The apparatus of claim 9, wherein the power supply includes a battery.

11. The apparatus of claim 9, wherein the power supply is self-contained.

12. The apparatus of claim 11, wherein the sensor device is adapted to generate sensor energy from the infusion fluid flow, the sensor energy being stored in the power supply.

13. The apparatus of claim 9, wherein the power supply includes a battery, and a charger adapted to charge the battery via inductive coupling.

14. The apparatus of claim 9, wherein the external RFID transceiver is adapted to selectively send an RF signal to the sensor device, the RF signal being operable to charge the power supply.

15. The apparatus of claim 1, further comprising a repeater disposed outside of the sensor device, the repeater being adapted to receive the sensor signal and send the sensor signal to the platform.

16. A phacoemulsification apparatus for emulsifying a lens in a capsular chamber in a patient's eye, comprising:
a handle including a tip, the tip being adapted to receive a flow of fluid and to perform emulsification when inserted in the capsular chamber;
a platform adapted to provide the flow of fluid to the handle and adapted to respond to a sensor signal;
a sensor being sized to be disposed within the capsular chamber and generate the sensor signal, the sensor signal being indicative of a current fluid pressure within the capsular chamber; and
a transmitter adapted to be disposed outside the capsular chamber and wirelessly receive the sensor signal from the sensor, the transmitter being adapted to transmit the sensor signal to the platform.

17. The apparatus of claim 17, wherein the sensor includes an RFID receiver, and the transmitter includes an RFID transceiver adapted to provide a query signal to the RFID receiver and wirelessly receive the sensor signal.

18. The apparatus of claim 18, wherein the RFID receiver is adapted to power the sensor.
